(19) United States
(12) Patent Application Publication
(10) Pub. No.: US 2024/0252331 A1
(43) Pub. Date: Aug. 1, 2024

(54) BRIM FOR PROSTHETIC SOCKET

(71) Applicant: Ossur Iceland ehf, Reykjavik (IS)

(72) Inventors: Oskar Thor LARUSSON, Reykjavik (IS); Einar HREINSSON, Reykjavik (IS)

(21) Appl. No.: 18/425,765

(22) Filed: Jan. 29, 2024

Related U.S. Application Data
(60) Provisional application No. 63/441,931, filed on Jan. 30, 2023.

(51) Int. Cl. A61F 2/78; A61F 2/80

(52) U.S. Cl. CPC A61F 2/7843; A61F 2/80

(57) ABSTRACT

A brim is provided for securing to a proximal end of a prosthetic rigid socket. The brim includes a cuff section arranged to flexibly extend about and upwardly or proximally from a peripheral proximal end of the socket; and an interface section defining a lower or distal portion of the brim and extending distally from the cuff section, the interface section adhering to the inner surface of the socket. A prosthetic socket system includes the socket and the brim.

### FIELD OF THE DISCLOSURE

The disclosure relates to prosthetic devices and, more particularly, to an improved brim attachable to a proximal end of a prosthetic socket.

### BACKGROUND

A typical prosthetic leg and foot include a socket, pylon, and foot. A socket is the portion of a prosthesis that fits around and envelops a residual limb or stump to which prosthetic components, such as a foot, are attached. The socket must fit closely to the residual limb to provide a firm connection and support but must also be sufficiently loose to allow for circulation. The socket comfortably transfers loads from the residual limb to the ground when combined with proper fitting.

The prosthetic socket is where the residual limb or stump of the amputated limb fits into place, thus turning the socket into the point of integration between the human tissue and the replacement prosthetic limb. A prosthetic socket provides a surface for weight-bearing, additional support while walking, and assists in the overall control of the other prosthetic components. If the socket is fitted poorly, it can lead to complications, including ongoing pain, sores, or even blisters on the residual limb. The socket may be rigid and capable of being sealed or creating a hypobaric chamber once donned, with a valve to expel air from the socket.

A prosthetic liner is arranged as an interface between the socket and the residual limb to increase comfort during use. Generally, the prosthetic liner includes an elastomeric body having a closed distal end and an open proximal end pulled or rolled over the residual limb. The prosthetic liner adheres to the residual limb surface. It acts as an interface between the residual limb and the socket and facilitates an easy transition in contact between the residual limb and the rigid components of the socket.

A brim may be on the prosthetic socket and can be substantially more flexible than the prosthetic socket. The brim may offer a transition from the rigidity of the prosthetic socket to the residual limb, particularly at the proximal peripheral edge of the socket, to enable greater comfort and reduce the appearance of blisters, pain, and sores on the soft tissue of the residual limb. In addition, the softness and resiliency of the brim, particularly when compared to the inherent rigidity of the prosthetic socket, may take up volume changes and slight movement of the residual limb at the interface between the socket and the residual limb by offering an intermediate component of support and connection.

The brim may be configured for being placed in tensioned and relaxed positions, thereby assisting the donning and continual wear of the socket (i.e., tensioned in use, relaxed for donning and doffing). The brim is preferably configured to secure about an upper or proximal perimeter of the socket and extend over, beyond, or secure to the inner and outer surfaces of the socket.

Prosthetic rigid sockets have been formed using various techniques, including plaster of Paris molds, computer modeling, vacuum forming, and other techniques known to prosthetists. A socket may be made according to U.S. Pat. No. 5,885,509, granted Mar. 23, 1999; U.S. Pat. No. 5,971,729, granted March Oct. 26, 1999; and U.S. Pat. No. 7,105,122, granted Sep. 12, 2006, each of which is incorporated herein by reference.

An example of a brim is found in U.S. Pat. No. 9,474,635, granted Oct. 25, 2016, and incorporated herein by reference. A drawback to known brims includes possible failure of the brim in tearing due to the mismatch of the flexibility of the brim and the rigidity of the socket, particularly at the interface between the proximal peripheral edge of the socket and the brim. In addition, as the socket is substantially rigid, the softer, flexible material forming the brim can lead to tearing as the weaker material. Likewise, a difficulty may occur when incorporating the brim into the prosthetic socket, as the brim may separate or delaminate from the inner surface of a socket over repeated use.

Accordingly, from at least the above observations, there is a need for a brim that is more durable than known brims while preserving the inherent soft and flexible properties of known brims. There is further a need for a brim that can offer better adherence to a rigid prosthetic socket as there is a mismatch in material properties and types of materials used to form a socket and a brim.

### SUMMARY

According to various embodiments, a brim is arranged to secure a prosthetic socket and configured for tensioned and relaxed positions. The brim is configured to secure about an upper or proximal perimeter of the socket and extend beyond or secure to the inner and outer surfaces.

A cuff section, a transition section, and an interface section collectively form a basic structure to the brim. The basic structure may be a monolithic structure formed by a mass of material. The basic structure may be soft, flexible, and elastic.

In an embodiment, the brim defines a reinforcement layer extending across a transition section between the cuff and interface sections of the brim. The reinforcement layer is complementary to the basic structure of the brim and preferably embedded in the basic structure. The reinforcement layer mitigates potential failure at the transition section without impeding the flexibility and softness of the cuff section on the user's residual limb.

The cuff section defines an upper portion of the brim and is arranged to extend about and upwardly from the socket opening, at which the reinforcement layer thereby reinforces the transition section at the socket opening. The cuff section is configured to move between tensioned and relaxed positions. In the tensioned position, the cuff section is arranged to fold away from the socket opening under the resistance of the reinforcement layer. In contrast, in the relaxed position, the cuff section is arranged to extend above the socket opening reinforced by the reinforcement layer and generally concentric with the outer surface of the socket. The cuff section is flexible relative to the socket, permitting distension relative to an axis of the socket under resistance by the reinforcement layer, such that the cuff section substantially flexes relative to the socket.

The interface section defines a lower portion of the brim and is arranged to extend along the inner surface of the socket, such that the reinforcement layer extends distally along or within the supplementary layer, at least in part or preferably in entirety. For example, the interface section may be arranged to extend flush against the inner surface of the socket, and the socket may include a recess extending into the thickness of the socket and relative to areas outside the recess. The interface section fits within the recess, and the inner surface of the socket, in combination with areas of the inner surface of the socket outside the recess, defines a continuous inner surface without interruption yet reinforced by the reinforcement layer.

A challenge with silicone materials, which is a preferred material for the basic structure of the brim, is that they may be challenging to adhere using an adhesive. In the past, it had been found that brims tend to detach from the socket.

A supplementary layer may be adhered to an outer surface of the brim, at least about or yet further entirely circumferentially about at least the interface section. The supplementary layer is preferably porous, at least partly through its outer side, thereby enabling resin from the socket to bleed through or create a firm and inseparable interlocking bond with the brim. The supplementary layer maintains constant contact with the socket and becomes inseparable from the socket once the resin cures while interlocking with the material forming the supplementary layer. Likewise, the material forming the basic structure of the brim interlocks from an inner side of the supplementary layer to form opposed sides of the supplementary layer interlocking with different materials to permanently secure the supplementary layer to both the basic structure of the brim and the socket.

By interlocking the resin of the socket to the silicone of the basic structure of the brim by the supplementary layer, an adhesive may be eliminated. Therefore, the interlocking creates a mechanical connection between the brim and the socket. However, there may be some chemical bonding between the silicone to the supplementary layer and the resin to the supplementary layer.

In the tensioned position, the cuff section may be arranged to fold downward away from the opening and generally over the outer surface of the socket. The cuff and interface sections may be formed integrally with one another from the same material. The brim may be formed by molding, such as injection molding, the basic structure of elastomeric material. The reinforcement layer may be embedded within a wall thickness of the brim so that it is inaccessible from the inner and outer sides of the brim.

The cuff section may define an asymmetrical proximal edge having a maximum height extending a greater distance from the socket opening than a minimum height of the upper edge, as measured from the transition section. The reinforcement layer may extend to a continuous height into the cuff section, measured from the distal peripheral edge, or be arranged asymmetrically relative to the asymmetrical proximal edge.

The maximum height may be at the first side of the socket, and the minimum height is at a side opposite the maximum height. The upper edge tapers from the maximum to the minimum height, measured from the periphery proximal edge to the transition section. The interface section may have a thickness smaller than the thickness of the cuff section, particularly since, according to a variation, the interface section may form part of the thickness of the socket, so the combined thickness of the interface section and the recessed section of the socket is the same as the thickness of the socket outside of the recessed section.

The brim may have various differing thicknesses to accommodate different degrees of flexibility and durability about the circumference of the brim. The proximal end of the brim may be thicker and taper, consistently or variably toward the distal end. The interface section may have a consistent, uniform height about the circumference of the brim or may vary, similarly to the variation of the upper section having an asymmetrical height from the transition section.

The transition section may include an indent about at least part of the periphery from the outer surface of the transition section into a thickness of the brim. The indent may be uniform or variable depending on its location in the periphery, partly due to the section of a residual limb by which the brim corresponds. A greater indent may be useful where there is greater weight bearing, corresponding in a thicker cuff section above such an indent (i.e., medial portion of the socket).

Numerous other advantages, features, and functions of embodiments of a brim will become readily apparent and better understood because of the following description and accompanying drawings.

### GLOSSARY

The terms set forth below will have the meanings as defined.

The term “anterior” has its ordinary meaning and refers to a location ahead of or to the front of another. The term “posterior” also has its ordinary meaning and refers to a location behind or to another location's rear.

The term “distal” has its ordinary meaning and refers to a location that is further from the heart than another location. The term “proximal” also has its ordinary meaning and refers to a location that is closer to the heart than another location.

The terms “rigid,” “flexible,” and “resilient” may distinguish characteristics of portions of certain features. The term “rigid” should denote that an element is generally devoid of flexibility. Within the context of features that are “rigid,” it should indicate that they do not lose their overall shape when force is applied and may break if bent with sufficient force. The term “flexible” should denote those features are capable of repeated bending such that the features may be bent into retained shapes or the features retain no general shape, but continuously deform when force is applied. The term “resilient” should denote that an element or feature that is not easily broken and may withstand continued use or movement.

The term “elastic” means that a structure is able to resume its normal shape spontaneously after contraction.

The term “generally is meant to connote “mostly” or “near exactly,” but not necessarily exactly, and refers to at least 90% as much.

The term “user” refers to a person who wears the socket. The user may be a patient or an operator. The term “clinician” refers to a clinical specialist, supervisor, therapist, doctor, or person with a similar role that assists or oversees the operation of the fabrication and observance of the socket by the user.

The term “monolithic” denotes a unitary structure, wherein the unitary structure is not provided by a chemical or mechanical fusion of multiple structures, but rather the unitary structure is formed from and defines a single mass of material consistently of the same type (for example, a structure formed by injection molding from an elastomer).

The term “anisotropic” denotes a physical property that has a different value when measured in different directions, for example the reinforcement layer strongly resists elongation in a longitudinal direction while being freely distendable laterally in a radial direction when it is embedded in the basic structure formed by the soft and elastic material of the brim.

It will be understood that, unless a term is defined to possess a described meaning, there is no intent to limit the meaning of such term, either expressly or indirectly, beyond its plain or ordinary meaning. The embodiments of the disclosure are adapted for a human body and may be dimensioned to accommodate different types, shapes, and sizes of human body sizes and contours.

These and other features, aspects, and advantages of the present disclosure will become better understood regarding the following description, appended claims, and accompanying drawings.

### BRIEF DESCRIPTION OF THE DRAWINGS

FIG. 1 is a perspective view of a prosthetic socket and a brim attached thereto.

FIG. 2 is a perspective view of the brim in FIG. 1.

FIG. 3 is a cross-sectional view taken along line III-III in FIG. 2.

FIG. 4 is a detail sectional view taken from detail IV in FIG. 3.

FIG. 5 is a detail sectional view taken from detail V in FIG. 3.

FIG. 6A is a schematic view showing the brim in a tensioned configuration for donning and doffing the socket.

FIG. 6B is a schematic view showing donning and doffing the socket in the tensioned configuration.

FIG. 7A is a schematic view showing moving the brim in a relaxed configuration from the tensioned configuration for use of the socket.

FIG. 7B is a schematic view showing placement of the brim and socket over the residual limb during use.

The drawing figures are not necessarily drawn to scale but instead are drawn to provide a better understanding of the components thereof and are not intended to be limiting in scope but to provide exemplary illustrations.

### DETAILED DESCRIPTION OF VARIOUS EMBODIMENTS

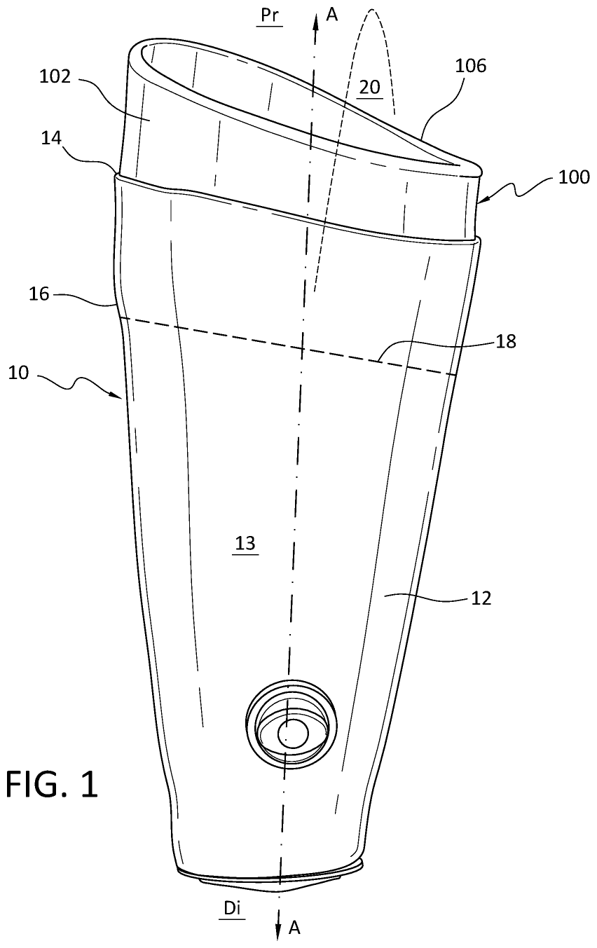

FIG. 1 illustrates a brim 100 securing to a proximal end of a prosthetic rigid socket 10. The socket 10 defines an opening 20 at the proximal end Pr, and outer and inner surfaces 13, 15. The brim 100 is coaxial to a central axis A-A of the socket, and preferably extends circumferentially about the proximal end of the socket 10. The brim 100 is generally tubular. The brim 100 has an opening 110 coaxial to the central axis A-A, and is open at proximal and distal ends Pr, Di of the brim 100.

The brim 100 has an outer surface 111 that is coextensive with the outer surface 15 of the socket wall 12, such that the brim 100 projects from the socket 10 generally along a same trajectory as the socket wall 12 extends in a proximal direction Pr. A slight bulge 16 may be apparent along the outer surface 15 of the socket wall 12 as the socket 10 accommodates the brim and to ensure an inner surface of the brim 112 is continuous with a contour or profile of the inner surface 15 of the socket. As shown in FIG. 1, bulge 16 may correspond to a circumferential or peripheral distal edge of the brim, demarcating its termination at line 18.

The brim includes a cuff section 102 arranged to flexibly extend about and upwardly or proximally from a peripheral proximal end 14 of the socket 10. An interface section 104 defines a lower or distal portion of the brim 100, and extends distally from the cuff section 102. The interface section 104 adheres to the inner surface 14 of the socket 10. The interface section 104 preferably is coextensive with the inner surface 14 of the socket 10 without interruption, in that the inner surface 112 and the interface section 104 are without interruption along their combined continuous profile as a single trajectory and without any discernable break or seam to the user.

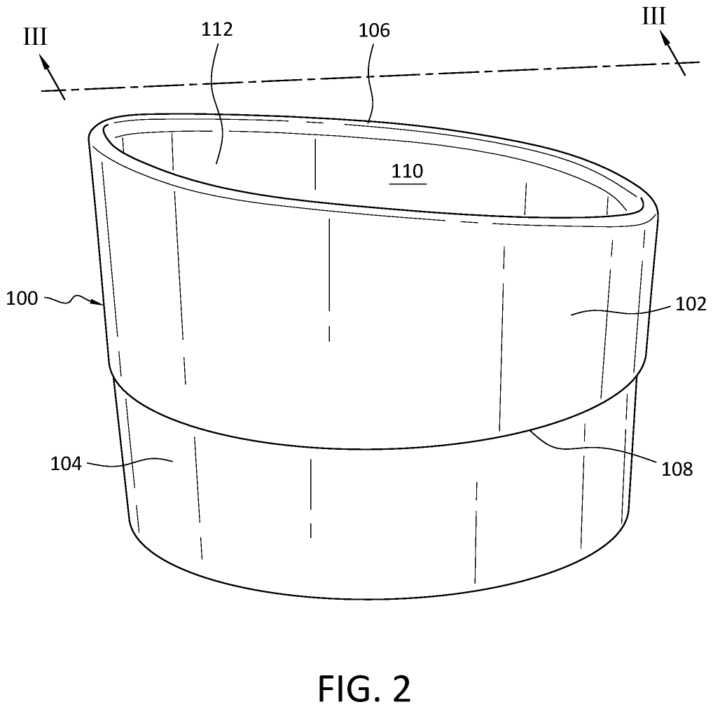

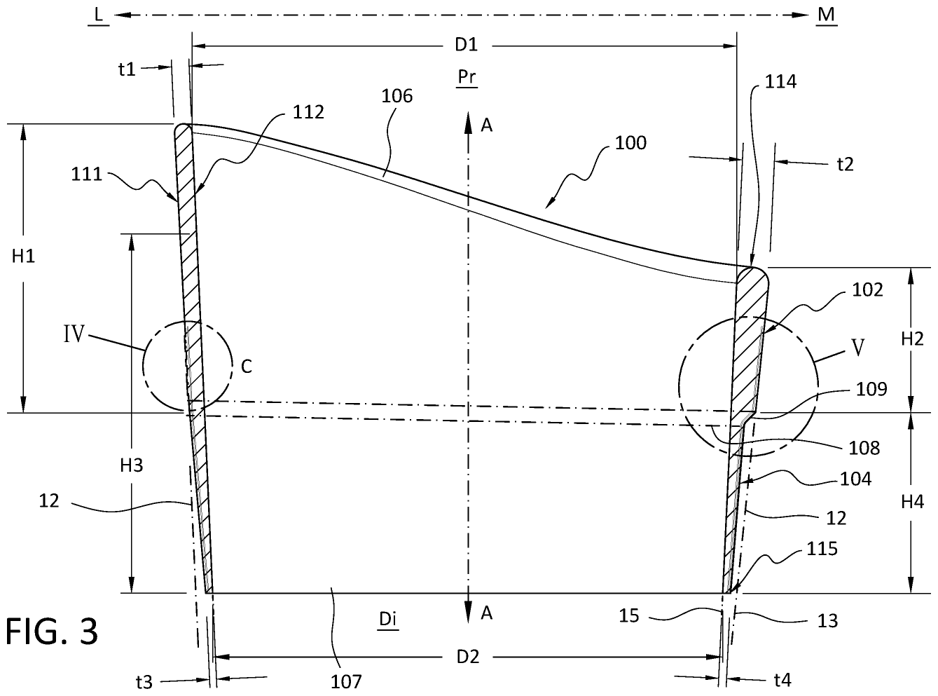

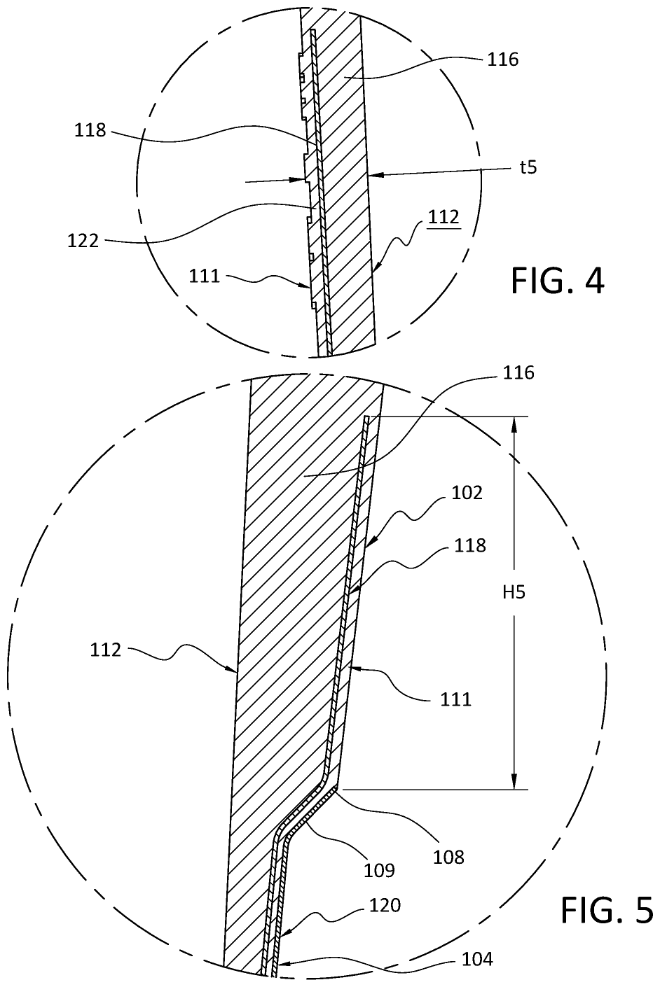

As shown in FIGS. 2 and 3, a transition section 108 is defined between the cuff section 102 and the interface section 104, and circumferentially demarcates the cuff section 102 from the interface section 104, at least along the outer surface 111 of the brim 100. The transition section 108 is located and corresponds to the socket where the brim attaches to the socket (i.e., interface section 104) and where the brim extends beyond the proximal peripheral edge of the socket (i.e., cuff section 102). The transition section 108 may only be present or distinguishable about or along the outer surface 111, whereas the inner surface 112 across the cuff section 102 and the interface 104 may be a continuous and generally linear (i.e., less than 1 mm deviation) surface.

The cuff section, interface section and the transition section may be formed as a monolithic structure and forms the basic structure of the brim in that these sections are molded from material. As will be explained, in addition to the basic structure of the brim, there may be a reinforcement layer embedded in the basic structure, and a supplementary layer that may be embedded or interlocked with the basic structure or at least a surface thereof.

The transition section 108 defines an indent 109 about at least a segment of an outer periphery 111 of the brim 100, particularly in an area whereby greater adherence or stability is required at the interface between the socket and the brim. For example, the indent 109 may correspond to a medial side M of the brim 100 whereby a greater load from the user may apply to the brim 100, whereas the lateral side L incurs a lighter load. The indent 109 may taper in size depending on the location about the circumference of the brim 100. The indent 109 is defined as a wall thickness reduction from the cuff section 102 to the interface section 104 along an outer surface 111 of the brim 100. The indent 109 may extend obliquely toward the axis A-A from the cuff section 102 to the interface section 104, as a gentle transition between the two sections.

A reinforcement layer 118 extends at least across the transition section 108 from the interface section 104 to the cuff section 102. The reinforcement layer 118 may be applied along the inner surface, the outer surface or within or partly within a wall thickness of the brim. According to the depicted embodiment in FIG. 3, the reinforcement layer 118 is embedded within a wall thickness 116 of the brim 100, and is fully between and within the outer and inner surfaces 111, 112 of the brim 100.

The reinforcement layer mitigates potential failure at the transition section, without impeding the flexibility and softness of the cuff section on the residual limb of the user. The reinforcement layer is preferably more rigid and less elastic than a material forming the transition section, as well as the cuff section and the interface section. Therefore, movement of the brim is resisted by the reinforcement layer, while the reinforcement layer permits such movement, the reinforcement layer enables repeated use without failure of the softer and more elastic or resilient material of the brim.

The reinforcement layer is preferably an elasticity-controlling matrix material relatively compliant in a radial direction and substantially rigid or inelastic in the axial direction, therefore the reinforcement layer is anisotropic. It follows that by decreasing the elongation in the soft silicone by the reinforcement layer, the reinforcement layer protects the transition section by protecting it from tears when subjected to radial movement during use. Any appropriate reinforcement matrix that would provide such properties could be used for the reinforcement layer, but as a practical matter a circular knit glass fiber or Nylon material is appropriate, provided it has the anisotropic properties described above.

According to a variation, the reinforcement layer 118 may be a substrate, textile or film with openings or a mesh that enables material forming at least the transition section to bled and impregnate the reinforcement layer, thereby the material forming the basic structure of the brim extends through the reinforcement layer. The reinforcement layer 118 may formed by a textile, and may be arranged near the outer outer surface 111 of the brim 100, in that it is at least 50% closer to the outer surface than the inner suface 112 within the wall thickness of the brim, and preferably 75% closer to the outer surface 111, and more preferably 85% closer to the outer surface 111.

As depicted, the reinforcement layer 118 extends from the peripheral or circumferential distal edge 107 of the interface section 104, through the transition section 108, and into a segment of the cuff section 102. According to a preferred embodiment, the reinforcement layer 118 extends approximately a quarter of a height H1, H2 of the cuff section 102, and depending on where about the circumference of the brim, the reinforcement layer 118 may extend about halfway (+ or −5%) of a height H2 of the brim, such as the medial side M. However, the extent by which the reinforcement layer 118 extends into the cuff section 102 may be governed by the extent reinforcement is required and can be anywhere between 0 to 100%.

It is preferable that the reinforcement layer extends at least past the transition section 108 into the cuff section 102 as the area of which the brim is most susceptible to tearing is where the brim transitions from attachment to the socket at the interface section and whereat it extends freely from the socket at the cuff section. As the cuff section 104 extends away from the transition section 108, the reinforcement layer 118 enables a gradual decrease in stiffness of the cuff section 102, whereby it is preferred the proximal end of the brim freely and flexibly extends from the socket, whereas the transition section offers stability for the user between the socket and the residual limb. The reinforcement layer, therefore, reduces the likelihood of tearing, providing light protection from tearing as it is completely enclosed by the material of the basic structure forming the brim yet preferably located beneath the outer surface.

For example, the reinforcement layer may be only needed at the transition section and may minimally extend into the cuff section and/or the interface section, for example such minimally extension can be less than 25 mm into the cuff section or the interface section. However, the reinforcement layer is not limited to minimal extend, and may extend entirely across either the cuff section or interface section.

The thickness t1, t2 of the cuff section 102 increases toward a circumferential or peripheral proximal edge 106 and may vary relative to the proximal end of the brim and the location about the circumference of the brim. As shown, the lateral side L has a thinner thickness t1 than the thickness t2 at the medial side M. A thickness t3, t4 of the interface section 104) decreases toward a distal edge 107. The indent 109 may bring the thickness t2 on the medial side M into line with the thickness t1 on the lateral side L so that the thickness of the interface is generally uniform, although the brim is not limited to such a configuration.

The proximal edge 106 of the brim 100 may be asymmetrical to accommodate better the residual limb and the way the socket rests on the user. For example, a segment about a circumference of the cuff section 102 on a first or lateral L side of the brim 100 has a greater height H1 from the transition section to the proximal edge 106 or corner 114 than a height H2 another segment of the cuff section 102 on a second or medial M side of the brim 100. The thickness of the cuff section 102 may likewise correspond to the height differences. For example, the cuff section 102 may have a greater wall thickness t2 between the outer and inner surfaces 111, 112 at the second or medial side M of the brim 100 than a wall thickness t1 at the first or lateral L side of the brim 100.

By creating the greater height H1 and corresponding thinner thickness t1, there is enhanced flexibility at least at such section by providing a longer section from the transition section. For example, but not limiting, H1 may be 150 mm, and H2 may be 120 mm, with a tolerance of at least 2.5 mm in either direction. Likewise, for example, the thickness t1 or lateral wall, may be 4.5 to 5.5 mm, whereas the maximum thickness t2 may be 8.5 to 10 mm.

The brim 100 may taper from the proximal to the distal edges 114, 115, in that the opening 110 has a proximal diameter D1 that is greater than the distal diameter D2, particularly as a residual limb may taper distally.

The interface section 104 preferably has a uniform height H3, H4 from the distal edge 107 or distal corner 115 to the transition section 108 about the circumference of the brim 100.

A supplementary layer 120 may be adhered to an outer surface of the interface section 104 and the transition section 108 to enhance engagement with the socket 10. The supplementary layer 120 may extend partially, in segments or entirely circumferentially about at least the interface section 104. The supplementary layer 120 may be a film, mesh or other type of layer that is porous or has interstices, such as a textile layer.

The supplementary layer 120 interlocks via the porosity thereof with material forming the brim, which bleeds through at least part of the supplementary layer 120 so that it is inseparable from the interface layer 104 and the transition section 108. Importantly, the outer side and at least a portion into the depth of the supplementary layer 120 are not impregnated by the material of the brim, leaving open for resin of the socket to bleed through the remainder of the thickness of the supplementary layer 120 to form a strong and inseparable bond with the socket.

The basic structure of the brim encompassing the cuff section 102, the interface section 104 and transition section 108 is formed by an elastomer, preferably silicone. Preferably, the cuff section 102, interface section 104 and the transition section 108 are formed and molded from a single silicone mass, forming a monolithic and unitary contiguous structure. Advantageously, the reinforcement layer 118 may be embedded within the material forming the basic structure so that it is fully concealed and embedded therein. Likewise, the supplementary layer 120 may be adhered to the basic structure before the final curing of the material forming the basic structure so that such material interlocks the structure of the supplementary layer 120.

The basic structure may be molded to include indicia 122, and form variable minor wall thickness t5 variants while still concealing and embedding the supplementary layer.

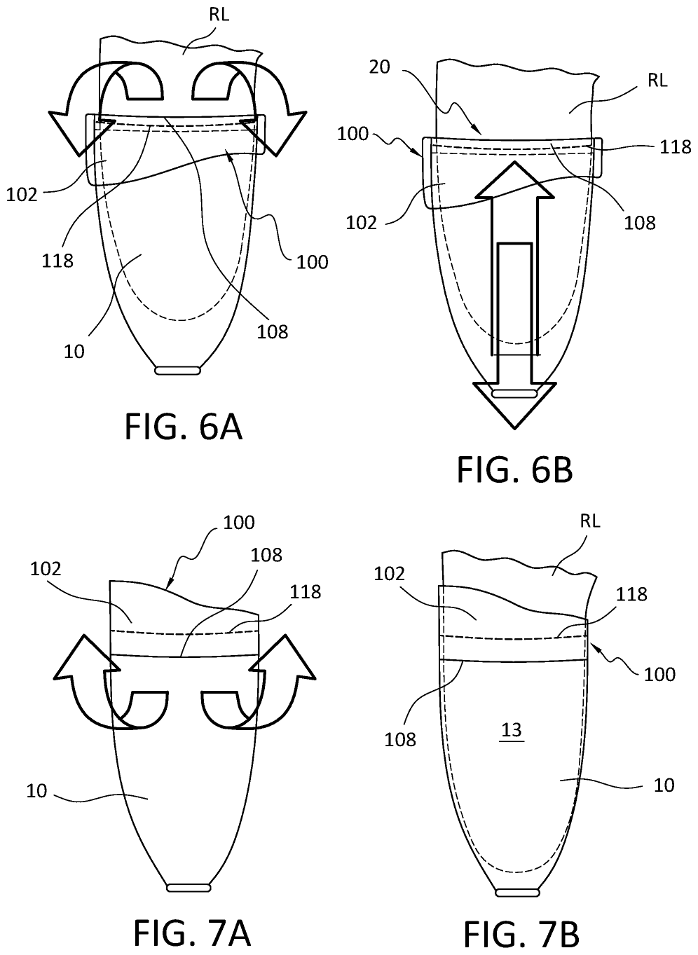

FIGS. 6A-6B illustrate how in the tensioned position, the cuff section 102 of the brim 100 may be arranged to fold downwardly away from the transition section 108 and generally over the outer surface of the socket to enable donning and doffing of the socket 10 by the residual limb RL. At least a portion of the reinforcement layer 118 is embedded in the cuff section 102 as the cuff section 102 folds over the socket 10, and the reinforcement layer 118 reinforces the durability of repeated placement of the cuff section 102 in the tensioned position.

FIGS. 7A-7B exemplify the cuff section 102 in the relaxed position such that the cuff section 102 is arranged to extend above or proximally from the socket opening 20 and generally concentric with the outer surface 13 of the socket 10. At least a portion of the reinforcement layer 118 remains above the transition section 108 in the cuff section 102.

It is to be understood that not necessarily all objects or advantages may be achieved under an embodiment of the disclosure. Those skilled in the art will recognize that a brim may be embodied or carried out, so it achieves or optimizes one advantage or group of advantages as taught herein without achieving other objects or advantages as taught or suggested herein.

The skilled artisan will recognize the interchangeability of various disclosed features. Besides the variations described, other known equivalents for each feature can be mixed and matched by one of ordinary skill in this art to build and use a prosthetic socket under the principles of the present disclosure. Furthermore, the skilled artisan will understand that the features described may be adapted to other methods and types of prosthetic devices.

Although this disclosure describes certain exemplary embodiments and examples of a brim for a prosthetic socket, it will be understood by those skilled in the art that the present disclosure extends beyond the specifically disclosed components to other alternative embodiments and/or uses of the disclosure and obvious modifications and equivalents thereof. It is intended that the present disclosure should not be limited by the disclosed embodiments described above and may be extended to other applications that may employ the features described.

1. A brim for securing to a proximal end of a prosthetic rigid socket, the socket defining an opening at the proximal end, and outer and inner surfaces, the brim being coaxial to a central axis of the socket, the brim comprising:
a cuff section arranged to flexibly extend about and upwardly or proximally from a peripheral proximal end of the socket;
an interface section defining a lower or distal portion of the brim extending distally from the cuff section, the interface section adhering to the inner surface of the socket;
a reinforcement layer corresponding at least with the interface section.

2. The brim of claim 1, wherein the reinforcement layer extends across a transition section extending from the interface section to the cuff section.

3. The brim of claim 1, wherein an inner surface of the brim is continuous and smoothly transitions from the cuff section to the interface section across the transition section without interruption and variation in contour.

4. The brim of claim 1, wherein the reinforcement layer is formed from a textile having less elasticity from a material forming the cuff section.

5. The brim of claim 1, wherein a transition section is defined between the cuff section and the interface section, wherein the transition section defines an indent about at least a segment of an outer periphery (of the brim.

6. The brim of claim 5, wherein the indent is defined as a reduction of wall thickness from the cuff section to the interface section along an outer surface of the brim.

7. The brim of claim 5, wherein a thickness of the cuff section increases toward a proximal circumferential edge.

8. The brim of claim 7, wherein a thickness of the interface section decreases toward a distal circumferential edge.

9. The brim of claim 8, wherein a segment about a circumference of the cuff section on a first or lateral side of the brim has a greater height from the transition section to the proximal circumferential edge than a height of another segment of the cuff section on a second or medial side of the brim.

10. The brim of claim 9, wherein the cuff section has a greater wall thickness between the outer and inner surfaces at the second or medial side of the brim than a wall thickness at the first or lateral side of the brim.

11. The brim of claim 9, wherein the interface section has a uniform height from the distal circumferential edge to the transition section about the circumference of the brim.

12. The brim of claim 1, wherein the cuff section is configured to move between a tensioned position and a relaxed position, wherein in the tensioned position the cuff section is arranged to fold downwardly away from the socket opening and generally over an outer surface of the socket, wherein in the relaxed position the cuff section is arranged to extend above or proximally from the socket opening and generally concentric with the outer surface of the socket.

13. The brim of claim 12, wherein the interface section defines a lower portion of the brim and is arranged to extend along a surface of the socket in both the tensioned and relaxed positions.

14. The brim of claim 1, wherein the interface section secures to the inner surface of the socket.

15. The brim of claim 1, further comprising a supplementary layer located about or along an outer surface of the interface section.

16. The brim of claim 15, wherein the supplementary layer has an inner surface interlocking or bonded to the outer surface of the interface section, the supplementary layer having an outer surface defining a mesh or porous structure extending at least partially through a thickness of the supplementary layer for engagement with resin of the socket.

17. A prosthetic socket system comprising:
a prosthetic rigid socket defining an opening at a proximal end, and outer and inner surfaces;
a brim securing to a proximal end of a prosthetic rigid socket, the brim being coaxial to a central axis of the socket, the brim further comprising:
a cuff section arranged to flexibly extend about and upwardly or proximally from a peripheral proximal end of the socket;
an interface section defining a lower or distal portion of the brim (100) extending distally from the cuff section (102), the interface section (104) adhering to the inner surface (14) of the socket (10).

18. The prosthetic socket system of claim 17, wherein the brim further comprises a reinforcement layer corresponding at least with the interface section;
wherein the reinforcement layer extends across a transition section from the interface section to the cuff section, and the transition section extends from the interface section to the cuff section.

19. The prosthetic socket system of claim 17, wherein the brim further comprises a transition section is defined between the cuff section and the interface section, wherein the transition section defines an indent about at least a segment of an outer periphery of the brim.

20. The prosthetic socket system of claim 17, wherein the brim includes a segment about a circumference of the cuff section on a first or lateral side of the brim has a greater height from the transition section to the proximal circumferential edge than a height another segment of the cuff section on a second or medial side of the brim.
